(19) United States
(12) Patent Application Publication
(10) Pub. No.: US 2006/0155125 A1
(43) Pub. Date: Jul. 13, 2006

(54) Synthetic process

(76) Inventors: Ping Chen, Belle Mead, NJ (US); Derek J. Norris, Pennington, NJ (US); Ashvinikumar V. Gavai, Princeton Junction, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/314,794

(22) Filed: Dec. 21, 2005

Related U.S. Application Data
(60) Provisional application No. 60/638,335, filed on Dec. 22, 2004.

(51) Int. Cl. C07D 487/02; C07D 487/04

(52) U.S. Cl. 544/184; 544/279; 544/280

(57) ABSTRACT

The present invention provides a process for preparing compounds of formula (IV) 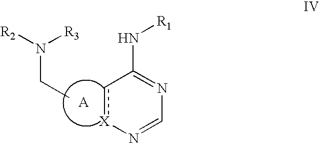 or a pharmaceutically acceptable salt thereof. The compounds prepared by the process of the invention inhibit tyrosine kinase activity of growth factor receptors such as HER1, HER2 and HER4 thereby making them useful as antiproliferative agents for the treatment of cancer and other diseases.

This application claims the priority benefit of U.S. Provisional Application No. 60/638,335 filed Dec. 22, 2004, which is incorporated herein by reference in its entirety.

### FIELD OF THE INVENTION

This invention relates to a novel, improved processes for the preparation of bicyclic aromatic compounds that inhibit the tyrosine kinase activity of growth factor receptors such as HER1, HER2, and HER4 thereby making them useful as anti-cancer agents. The compounds prepared by the processes of the invention are also useful in the treatment of diseases, other than cancer, which are associated with signal transduction pathways operating through growth factor receptors such as HER1, HER2 and HER4.

### SUMMARY OF THE INVENTION

The present invention provides an improved process for the preparation of bicyclic aromatic compounds (I) and a key intermediate (Compound 9) for the preparation thereof. Compounds such as those disclosed in copending U.S. Provisional Patent Applications 60/533,335 and 60/533,361 filed Dec. 29, 2003 and 60/620,784 filed Oct. 21, 2004 are included. The disclosures of said applications are hereby incorporated by reference in their entirety.

In one embodiment, the improved process of the invention allows for a one pot, regioselective method for functionalizing two positions of the bicyclic nucleus at the 4 and 5 positions.

In a second embodiment, the invention provides processes for preparing a key intermediate that is amenable to large scale preparations and provides derivatives of high quality and significantly higher yield than previous processes.

### DETAILED DESCRIPTION OF THE INVENTION

The present invention provides a process for the preparation of compounds of the formula 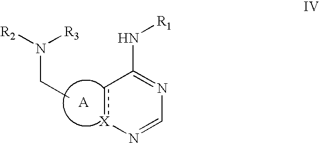
wherein

the A ring is a 5 or 6-membered carbocyclic ring optionally containing one or more heteroatom in the ring selected from —N—, —O— and —S—, with the proviso that when X is 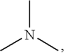
the A ring is a 5-membered aromatic, heterocyclic ring; provided that the compound formed is chemically stable;

R¹ is alkyl or substituted alkyl, aryl or substituted aryl, heteroaryl or substituted heteroaryl or heterocyclyl or substituted heterocyclyl;

R² and R³ are independently hydrogen, alkyl or substituted alkyl, alkoxy or substituted alkoxy, aryl or substituted aryl, heteroaryl or substituted heteroaryl or heterocyclyl or substituted heterocyclyl; or

R² and R³ are taken together with the nitrogen atom to form an optionally substituted 5-7 membered heterocyclic ring optionally containing one or more additional heteroatoms in the ring selected from —N—, —O— and —S—, provided that the compound formed is chemically stable;

X is 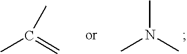
or a pharmaceutically acceptable salt or stereoisomer thereof,

which comprises the steps of

reacting Compound I of the formula 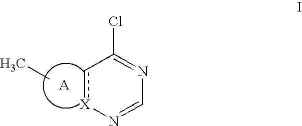
with N-bromosuccinimide in the presence of an activating agent, followed by treatment with a trialkylamine of the formula (R)₃N,
to afford Compound II of the formula 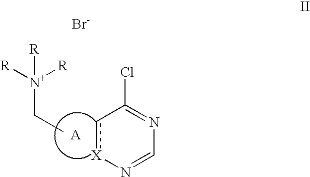
where R is C₁-C₄ alkyl,
which is subsequently reacted with a primary amine of the formula —R¹NH₂,
where R¹ is as defined above,
to afford Compound III of the formula 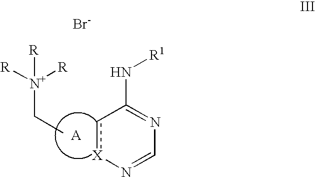
or a salt thereof,
where R¹ is as defined above,
which is reacted with a nucleophile of the formula 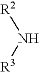
where R² and R³ are as previously defined,
in the presence of a base to afford Compound IV.

In another embodiment, Compound 1 of the formula 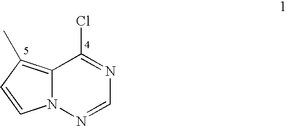
is reacted with N-bromosuccinimide in the presence of an activating agent, followed by treatment with a trialkylamine of the formula (R)₃N,
to afford Compound 2 of the formula 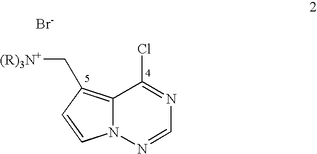
where R is C₁-C₄ alkyl,
which is subsequently reacted with a primary amine of the formula —R¹NH₂,
where R¹ is as defined above,
to afford Compound 3 of the formula 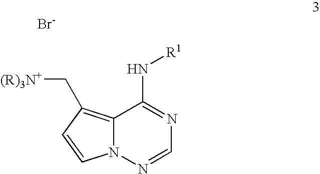
as a HCl salt,
which is reacted with a nucleophile of the formula 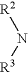
where R² and R³ are as previously defined,
in the presence of a base to afford Compound 4 of the formula

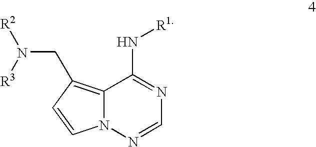

In a third embodiment, Compound 5 of the formula 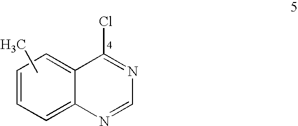
is reacted with N-bromosuccinimide in the presence of an activating agent, followed by treatment with a trialkylamine of the formula (R)₃N, to afford Compound 6 of the formula 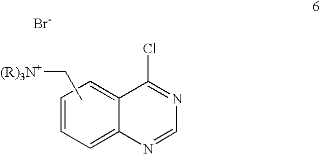
where R is C₁-C₄ alkyl,
which is subsequently reacted with a primary amine of the formula —R¹NH₂,
where R¹ is as defined above, to afford Compound 7 of the formula 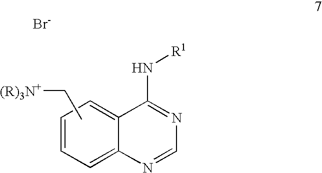
as a HCl salt,
which is reacted with a nucleophile of the formula 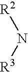
where R² and R³ are as previously defined,
in the presence of a base to afford Compound 8 of the formula

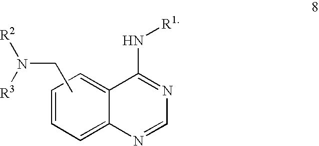

The invention also provides Compound 9 of the formula 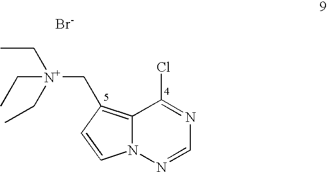
or a pharmaceutically acceptable salt thereof,
which is a key intermediate in the preparation of Compound 4.

The invention also provides a pharmaceutical composition comprising a compound of formula I and a pharmaceutically acceptable carrier, prepared by the process of the invention.

The invention also provides a pharmaceutical composition prepared by the process of the invention comprising a compound of formula I in combination with pharmaceutically acceptable carrier and an anti-cancer or cytotoxic agent. In one embodiment said anti-cancer or cytotoxic agent is selected from the group consisting of linomide; inhibitors of integrin αvβ3 function, angiostatin, razoxane, tamoxifen, toremifene, raloxifene, droloxifene, iodoxifene, megestrol acetate, anastrozole, letrozole, borazole, exemestane, flutamide, nilutamide, bicalutamide, cyproterone acetate, gosereline acetate, leuprolide, finasteride, metalloproteinase inhibitors, inhibitors of urokinase plasminogen activator receptor function, growth factor antibodies, growth factor receptor antibodies such as Avastin® (bevacizumab) and Erbitux® (cetuximab), tyrosine kinase inhibitors, serine/threonine kinase inhibitors, methotrexate, 5-fluorouracil, purine, adenosine analogues, cytosine arabinoside, doxorubicin, daunomycin, epirubicin, idarubicin, mitomycin-C, dactinomycin, mithramycin, cisplatin, carboplatin, nitrogen mustard, melphalan, chlorambucil, busulphan, cyclophosphamide, ifosfamide, nitrosoureas, thiotepa, vincristine, vinflunine, Taxol® (paclitaxel), Taxotere® (docetaxel), epothilone analogs, discodermolide analogs, eleutherobin analogs, etoposide, teniposide, amsacrine, topotecan, irinotecan, flavopyridols, biological response modifiers and proteasome inhibitors such as Velcade® (bortezomib), Iressa®, Tarceva® and Gleevec®.

The following are definitions of terms that may be used in the present specification. The initial definition provided for a group or term herein applies to that group or term throughout the present specification individually or as part of another group, unless otherwise indicated.

The term “alkyl” refers to straight or branched chain unsubstituted hydrocarbon groups of 1 to 20 carbon atoms, preferably 1 to 7 carbon atoms. The expression “lower alkyl” refers to unsubstituted alkyl groups of 1 to 4 carbon atoms.

The term “substituted alkyl” refers to an alkyl group substituted by, for example, one to four substituents, such as, halo, hydroxy, alkoxy, oxo, alkanoyl, aryloxy, alkanoyloxy, amino, alkylamino, arylamino, aralkylamino, disubstituted amines in which the 2 amino substituents are selected from alkyl, aryl or aralkyl; alkanoylamino, aroylamino, aralkanoylamino, substituted alkanoylamino, substituted arylamino, substituted aralkanoylamino, thiol, alkylthio, arylthio, aralkylthio, alkylthiono, arylthiono, aralkylthiono, alkylsulfonyl, arylsulfonyl, aralkylsulfonyl, sulfonamido, e.g. SO₂NH₂, substituted sulfonamido, nitro, cyano, carboxy, carbamyl, e.g. CONH₂, substituted carbamyl e.g. CONHalkyl, CONHaryl, CONHaralkyl or cases where there are two substituents on the nitrogen selected from alkyl, aryl or aralkyl; alkoxycarbonyl, aryl, substituted aryl, guanidino and heterocyclyls, such as, indolyl, imidazolyl, furyl, thienyl, thiazolyl, pyrrolidyl, pyridyl, pyrimidyl and the like. Where noted above where the substituent is further substituted it will be with alkyl, alkoxy, aryl or aralkyl.

The term “halogen” or “halo” refers to fluorine, chlorine, bromine and iodine.

The term “aryl” refers to monocyclic or bicyclic aromatic hydrocarbon groups having 6 to 12 carbon atoms in the ring portion, such as phenyl, naphthyl, biphenyl and diphenyl groups, each of which may be substituted.

The term “aralkyl” refers to an aryl group bonded directly through an alkyl group, such as benzyl.

The term “substituted aryl” refers to an aryl group substituted by, for example, one to four substituents such as alkyl, substituted alkyl, halo, trifluoromethoxy, trifluoromethyl, hydroxy, alkoxy, alkanoyl, alkanoyloxy, amino, alkylamino, aralkylamino, dialkylamino, alkanoylamino, thiol, alkylthio, ureido, nitro, cyano, carboxy, carboxyalkyl, carbamyl, alkoxycarbonyl, alkylthiono, arylthiono, arylsulfonylamine, sulfonic acid, alkysulfonyl, sulfonamido, aryloxy and the like. The substituent may be further substituted by hydroxy, alkyl, alkoxy, aryl, substituted aryl, substituted alkyl or aralkyl.

The term “heteroaryl” refers to an optionally substituted, aromatic group for example, which is a 4 to 7 membered monocyclic, 7 to 11 membered bicyclic, or 10 to 15 membered tricyclic ring system, which has at least one heteroatom and at least one carbon atom-containing ring, for example, pyridine, tetrazole, indazole, indole.

The term “alkenyl” refers to straight or branched chain hydrocarbon groups of 2 to 20 carbon atoms, preferably 2 to 15 carbon atoms, and most preferably 2 to 8 carbon atoms, having one to four double bonds.

The term “substituted alkenyl” refers to an alkenyl group substituted by, for example, one to two substituents, such as, halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino, indolyl, imidazolyl, furyl, thienyl, thiazolyl, pyrrolidyl, pyridyl, pyrimidyl and the like.

The term “alkynyl” refers to straight or branched chain hydrocarbon groups of 2 to 20 carbon atoms, preferably 2 to 15 carbon atoms, and most preferably 2 to 8 carbon atoms, having one to four triple bonds.

The term “substituted alkynyl” refers to an alkynyl group substituted by, for example, a substituent, such as, halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino and heterocyclyl, e.g. imidazolyl, furyl, thienyl, thiazolyl, pyrrolidyl, pyridyl, pyrimidyl and the like.

The term “cycloalkyl” refers to an optionally substituted, saturated cyclic hydrocarbon ring systems, preferably containing 1 to 3 rings and 3 to 7 carbons per ring which may be further fused with an unsaturated C₃-C₇ carbocylic ring. Exemplary groups include cyclopropyl, cyclobutyl, cyclopentyl, cyclohexyl, cycloheptyl, cycloctyl, cyclodecyl, cyclododecyl, and adamantyl. Exemplary substituents include one or more alkyl groups as described above, or one or more groups described above as alkyl substituents.

The terms “heterocycle”, “heterocyclic” and “heterocyclyl” refer to an optionally substituted, fully saturated or unsaturated, aromatic or nonaromatic cyclic group, for example, which is a 4 to 7 membered monocyclic, 7 to 11 membered bicyclic, or 10 to 15 membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized and the nitrogen heteroatoms may also optionally be quaternized. The heterocyclic group may be attached at any heteroatom or carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxazepinyl, azepinyl, 4-piperidonyl, pyridyl, N-oxo-pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, dioxanyl, isothiazolidinyl, thietanyl, thiiranyl, triazinyl, and triazolyl, and the like.

Exemplary bicyclic heterocyclic groups include 2,3-dihydro-2-oxo-1H-indolyl, benzothiazolyl, benzoxazolyl, benzothienyl, quinuclidinyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl (such as furo[2,3-c]pyridinyl, furo[3,1-b]pyridinyl] or furo[2,3-b]pyridinyl), dihydroisoindolyl, dihydroquinazolinyl (such as 3,4-dihydro-4-oxo-quinazolinyl), benzisothiazolyl, benzisoxazolyl, benzodiazinyl, benzimidazolyl, benzofurazanyl, benzothiopyranyl, benzotriazolyl, benzpyrazolyl, dihydrobenzofuryl, dihydrobenzothienyl, dihydrobenzothiopyranyl, dihydrobenzothiopyranyl sulfone, dihydrobenzopyranyl, indolinyl, indolyl, isochromanyl, isoindolinyl, naphthyridinyl, phthalazinyl, piperonyl, purinyl, pyridopyridyl, quinazolinyl, tetrahydroquinolinyl, thienofuryl, thienopyridyl, thienothienyl, and the like.

Exemplary substituents include one or more alkyl or aralkyl groups as described above or one or more groups described above as alkyl substituents.

Also included are smaller heterocyclyls, such as, epoxides and aziridines.

The term “heteroatoms” shall include oxygen, sulfur and nitrogen.

The compounds of formula I may form salts which are also within the scope of this invention. Pharmaceutically acceptable (i.e. non-toxic, physiologically acceptable) salts are preferred, although other salts are also useful, e.g., in isolating or purifying the compounds of this invention.

The compounds of formula I may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The compounds for formula I may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art.

In addition, zwitterions (“inner salts”) may be formed.

All stereoisomers of the compounds of the instant invention are contemplated, either in admixture or in pure or substantially pure form. The definition of compounds according to the invention embraces all the possible stereoisomers and their mixtures. It very particularly embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

It should further be understood that solvates (e.g., hydrates) of the compounds of formula I are also with the scope of the present invention. Methods of solvation are generally known in the art.

The pharmaceutical compositions of the present invention containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropyl-cellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisole or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solution. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS.™, model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouthwashes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a human subject, the daily dosage will normally be determined by the prescribing physician with the dosage generally varying according to the age, weight, sex and response of the individual patient, as well as the severity of the patient's symptoms.

If formulated as a fixed dose, such combination products employ the compounds of this invention within the dosage range described above and the other pharmaceutically active agent or treatment within its approved dosage range. Compounds of formula I may also be administered sequentially with known anticancer or cytotoxic agents when a combination formulation is inappropriate. The invention is not limited in the sequence of administration; compounds of formula I may be administered either prior to or after administration of the known anticancer or cytotoxic agent(s).

### Methods of Preparation

All temperatures are in degrees Celsius (° C.) unless otherwise indicated. Preparative Reverse Phase (RP) HPLC purifications were done on C18 reverse phase (RP) columns YMC S5 ODS columns eluting with 90% aqueous methanol containing 0.1% TFA as buffer solution and monitoring at 220 nm. For analytical HPLC 0.2% phosphoric acid was used instead of TFA. All of the synthesized compounds were characterized by at least proton NMR and LC/MS. During work up of reactions, the organic extract was dried over magnesium sulfate (MgSO₄), unless mentioned otherwise.

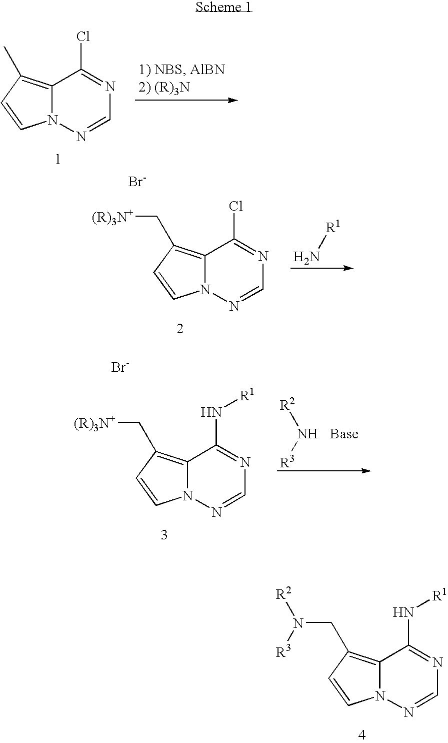

A general reaction scheme for the preparation of Compound 2 (N-((4-chloropyrrolo[1,2-f][1,2,4]triazine-5-yl)methyl-N,N,N-triethylammonium bromide) and its application, as a versatile synthetic intermediate, in the preparation of various pharmaceutically useful kinase inhibitors, such as Compound 4, is described in Scheme 1.

Compound 1 can be converted to Compound 2 by a two-step, one-pot procedure. Thus, reaction of Compound 1 with N-bromosuccinimide (NBS) in the presence of a radical initiator, such as 2,2′-azobisisobutyronitrile (AIBN) or benzoyl peroxide (Bz₂O₂), gives the intermediate, 5-(bromomethyl)-4-chloropyrrolo[1,2-f][1,2,4]triazine. Reaction of this intermediate with a trialkyl amine, such as triethlyamine, provides Compound 2 as a trialkyl ammonium salt in good overall yield. Generally, the reaction takes place in the presence of a solvent such as acetonitrile, carbon tetrachloride, tetrahydrofuran, chloroform, mixtures of cyclohexanes, chlorobenzene, dimethylformamide and dimethylacetamide.

Compound 2 is a versatile synthetic intermediate. Introduction of various functional groups to Compound 2 can be achieved in the following manner. Reaction of Compound 2 with a primary amine, in stoichiometric amount, gives compound 3, as a HCl salt, which can be further reacted with a nucleophile, in the presence or absence of a base, such as a tertiary amine, to give the desired Compound 4.

As an extension, the synthetic process for Compound 2 and its application (Scheme 1) can be applied to other heterobicyclic systems, such as quinazolines, represented by 5, or 6-, or 7-((dialkylamino)methyl)-N-aryl-quinazolin-4-amine, Compound 6. In addition, Compound 6 can be used in the preparation of various pharmaceutically useful kinase inhibitors, represented by Compound 8 (Scheme 2).

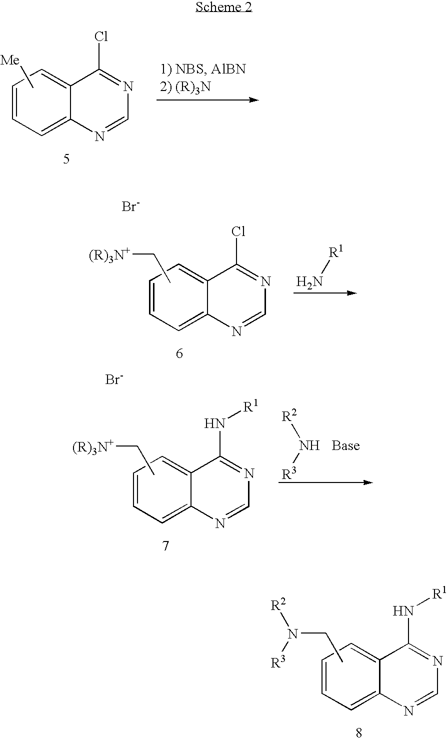

### EXAMPLE 1

### Synthetic Procedure for Preparation of Compound 2 (where R=Et):

Preparation of Compound 10:

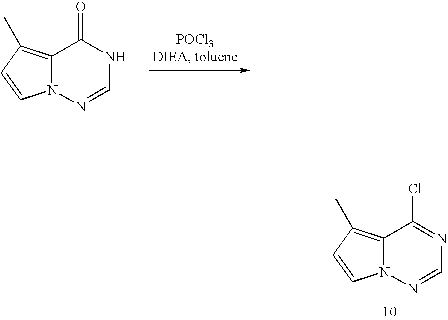

To 35.45 g of 5-methylpyrrolo[1,2-f][1,2,4]triazin-4(3H)-one (MW=149.15, 0.238 mol) in 500 mL of toluene under nitrogen was added (in one portion) 28 mL of phosphorus oxychloride (MW=153.33, 46.06 g, 0.300 mol, 1.26 eq), followed by 33 mL of N,N-diisopropylethylamine (MW=129.25, 24.49 g, 0.189 mol, 0.79 eq). The reaction mixture was heated from RT to reflux for 5.5 h. HPLC analysis showed that the reaction was complete. The reaction mixture was allowed to cool to RT overnight. The toluene was removed under reduced pressure by rotary evaporation. The residue was dissolved in 700 mL of methylene chloride and washed with ice-cold saturated aqueous sodium bicarbonate solution (in the presence of ice). The aqueous layer was extracted once with 200 mL of methylene chloride. The combined organic layers were dried over anhydrous magnesium sulfate. Upon filtration and evaporation under reduced pressure, the residue was passed through a short silica gel column (˜5 inches high), using methylene chloride as the eluent. Evaporation of solvent under reduced pressure gave 31.92 g (80% yield) of Compound 10 as a solid (MW=167.60).

### EXAMPLE 2

Preparation of Compound 9:

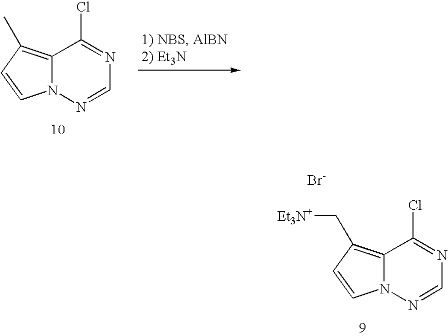

To a 2-liter round bottom flask was added 31.82 g (0.190 mol) of Compound 10, 3.38 g of AIBN (MW=177.99, 0.019 mol, 0.1 eq), and 35.85 g of NBS (MW=164.21, 0.218 mol, 1.15 eq). The reagents were placed under nitrogen and 900 mL of carbon tetrachloride was added. The mixture was degassed under vacuum and the nitrogen was reintroduced. This process was repeated twice. The mixture was then heated from RT to 80° C. for 1 h. Upon cooling to room temperature, the solid was removed by filtration and the solid cake was rinsed with an additional 100 mL of carbon tetrachloride. The combined organic layers were washed with cold (˜10° C.) and dilute sodium bicarbonate (10% sat. diluted up to 500 mL), followed by cold brine and dried over anhydrous sodium sulfate. The reaction mixture was concentrated under reduced pressure and the resulting solid was dissolved in 300 mL of THF. 60 mL of triethylamine was added and the mixture was allowed to stir overnight. The newly formed solid was collected by filtration, and washed with an additional 200 mL of THF, followed by 200 mL of diethyl ether. The solid cake was dried under a steady flow of nitrogen for 30 min, and further dried under high vaccum for several hours. The resulting powder was stirred with 200 mL of anhydrous diethyl ether and 200 mL of anhydrous acetonitrile (CH₃CN) for 2 hours under nitrogen. The solid was then collected by filtration and rinsed with ˜200 mL of anhydrous diethyl ether. Drying under a steady nitrogen flow followed by under high vacuum yielded 49.66 g (75% overall from Compound 10) of compound 9 as a powder.

Alternative Preparation of Compound 9:

A solution of Compound 10, 4-chloro-5-methyl pyrrolotriazine, (560 mg, 3.34 mmol) in a mixture of chlorobenzene and cyclohexane (8.3 mL/8.3 mL) in a 100 mL round bottom flask was purged twice with nitrogen. AIBN (54 mg, 0.33 mmol) was added followed by the addition of NBS (692 mg, 3.84 mmol) at room temperature. The reaction mixture was submerged in an 80° C. oil bath and heated for one hour then cooled to room temperature. The major product was the desired 5-brominated compound (73%, as determined by the corresponding 5-methoxy compound, Rₜ=2.43 minute, 2% 7-brominated compound, and 9% starting material was recovered). Triethylamine (5 mL) was added and the reaction mixture was stirred for 14 hours, becoming heterogeneous. THF (15 mL) was introduced into the above mixture and stirred vigorously for 30 minutes. The solid was filtered, and washed with THF to give Compound 9 as a solid (1.07 g, 2.45 mmol, 73% yield) with about 92% purity by HPLC. Note: HPLC conditions: YMC S5 ODS 4.6×50 mm, 10-90% aqueous methanol containing 0.2% H₃PO₄, 4 min gradient, monitored at 220 nm on Shimadzu SCL 10A system.

### EXAMPLE 3

Preparation of Compound 11:

### [4-(3-chloro-4-fluoro-phenylamino)pyrrolo[2,1-f][1,2,4]triazin-5-ylmethyl]-triethyl-ammonium bromide hydrochloride

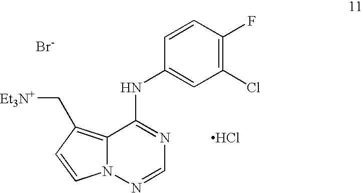

A mixture of Compound 9 (1.0 g, 2.2 mmol) and 3-chloro-4-fluoro-phenylamine (418 mg, 2.87 mmol) in CHCl₃ (10 ml) was heated at 50° C. for 2 h. The solid was filtered and rinsed with CHCl₃ and dried to give compound 11 (1.24 g, 87.4%). The compound had an analytical HPLC retention time=2.19 min. (Chromolith SpeedROD 4.6×50 mm, 10-90% aqueous methanol over 4 minutes containing 0.1% TFA, 4 ml/min, monitoring at 220 nm) and a LC/MS M⁺=376.
Preparation of Compound 12:

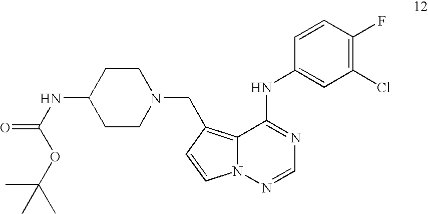

To a suspension of piperidin-4-yl-carbamic acid tert-butyl ester (4.1 g, 20.3 mmol) in CH₃CN (55 ml) at 70° C. was added a mixture of 11 (9.1 g, 18.4 mmol) and DIPEA (3.2 ml, 18.4 mmol) in CH₃CN (40 ml) dropwise in a period of 40 min. The reaction mixture was stirred at 70° C. for 1 h, then cooled to rt, after which H₂O (155 ml) was added slowly. The solid was filtered and rinsed with 15% CH₃CN/H₂O, then H₂O, and dried under vacuum to give 12 (7.84 g, 90%). The compound had an analytical HPLC retention time=2.73 min. (Chromolith SpeedROD 4.6×50 mm, 10-90% aqueous methanol over 4 minutes containing 0.1% TFA, 4 ml/min, monitoring at 220 nm) and a LC/MS M⁺+1=475.

### EXAMPLE 4

### 5-[(4-Amino-1-piperidinyl)methyl]-N-4-pyridinylpyrrolo[2,1-f][1,2,4]triazin-4-amine

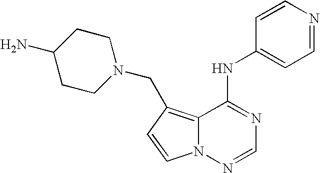

To a mixture of pyridin-4-ylamine (34 mg, 0.361 mmol) in THF (500 μl) was added 1N NaHMDS in THF (722 μl, 0.722 mmol). The mixture was cooled to 0° C. and a suspension of 9 (125 mg, 0.27 mmol) in DMF (800 μl) was added. The mixture was stirred at this temperature for 0.5 h. and piperidin-4-yl-carbamic acid tert-butyl ester (144 mg, 0.72 mmol) was added to the cold mixture. The reaction mixture was heated to 50° C. for 10 min and concentrated to remove THF. TFA (1 ml) was added and the mixture was stirred until the protecting group was removed (2 h). TFA was removed in vacuo and saturated NaHCO₃ was added. The mixture was extracted with EtOAc and the combined extracts were dried, concentrated and triturated with Et₂O to give the title compound (46 mg, 53%). Analytical HPLC retention time=0.51 min (Chromolith SpeedROD 4.6×50 mm, 10-90% aqueous methanol over 4 minutes containing 0.1% TFA, 4 ml/min, monitoring at 220 nm) and a LC/MS M⁺+1=324.

### EXAMPLES 5-8

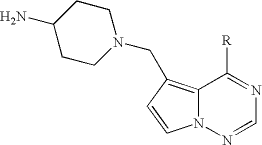

Compounds 5-8 were prepared using a similar process as the compound in Example 4 utilizing the corresponding amines.

| | | | | HPLC |
|---|---|---|---|---|
| | | | | Ret Time |
| Ex. # | R | Compound Name | [M + H] | (min) |
| 5 | 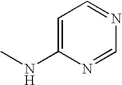 | 5-[(4-Amino-1- piperidinyl)methyl]-N- 4- pyrimidinylpyrrolo[2,1- f] [1,2,4]triazin-4-amine | 325 | 0.65 (b) |
| 6 | 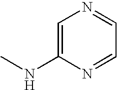 | 5-[(4-Amino-1- piperidinyl)methyl]-N- pyrazinylpyrrolo[2,1- f] [1,2,4]triazin-4-amine | 325 | 1.02 (b) |
| 7 | 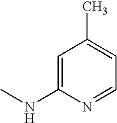 | 5-[(4-Amino-1- piperidinyl)methyl]-N- (4-methyl-2- pyridinyl)pyrrolo[2,1- f] [1,2,4]triazin-4-amine | 338 | 0.71 (b) |
| 8 | 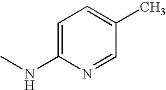 | 5-[(4-Amino-1- piperidinyl)methyl]-N- (5-methyl-2- pyridinyl)pyrrolo[2,1- f] [1,2,4]triazin-4-amine | 338 | 0.80 (b) |

### EXAMPLES 9-43

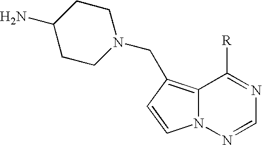 Method One

Compounds (with HPLC note (a)) were prepared by the following standard method.

In a 1 dram vial was added Compound 9 (55.0 mg, 0.16 mmol), aniline (0.16 mmol, 1.0 eq) and CH₃CN (1 ml). The mixture was shaken at 65° C. overnight. To this mixture was added piperidin-4-yl-carbamic acid tert-butyl ester (34.9 mg, 0.17 mmol) followed by addition of DIEA (28 μl, 0.16 mmol). The reaction was continued at 65° C. for 3 h. The mixture was concentrated; the residue was purified by Prep HPLC, and the desired fraction was collected and concentrated. The obtained residue was dried under high vacuum overnight.

To the above residue was added CH₂Cl₂ (1.5 ml) and TFA (0.2 ml), and the reaction mixture was shaken at rt for 2 h. The mixture was concentrated, and dried in speed vacuum overnight to give the solid product. Further Prep HPLC was used only when the solid was impure.

Method Two

Compounds (with HPLC note (b)) were prepared by the following standard method.

A mixture of Compound 9 (75 mg, 0.216 mmol) and anilines (1.0 eq, 0.216 mmol) in N,N-dimethyl acetamide (0.5 ml) in a small vial was heated at 70° C. for 3-5 hrs until a clear solution was obtained. HPLC was used to follow the progress of the reaction. The reaction mixture was cooled to rt and piperidin-4-yl-carbamic acid tert-butyl ester (43 mg, 0.216 mmol) was added, followed by the addition of N,N-diisopropylethylamine (75 μl). The reaction mixture again was heated to 70° C. overnight. Upon cooling, the reaction mixture was diluted with CH₂Cl₂ (0.5 ml) and cooled to 0° C. TFA (1.0 ml) was added and the mixture was stirred at ambient temperature overnight. The solvent was removed under reduced pressure (speedVac) and the residue was taken into methanol and purified by Prep HPLC to give the desired product.

| | | | | HPLC |
|---|---|---|---|---|
| | | | | Ret Time |
| Ex. | R | Compound Name | [M + H] | (min) |
| 9 | 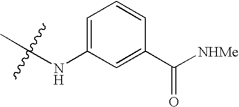 | 3-[[5-[(4-Amino-1- piperidinyl)methyl]pyrrolo[2,1- f] [1,2,4]triazin-4- yl]amino]-N-methyl benzamide | 380.25 | 0.91 (a) |
| 10 | 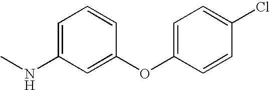 | 5-[(4-Amino-1- piperidinyl)methyl]-N- [3-(4- chlorophenoxy)phenyl]pyrrolo[2,1- f] [1,2,4]triazin-4-amine | 449.20 | 2.59 (a) |
| 11 | 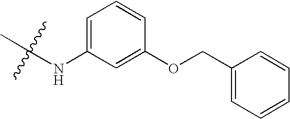 | 5-[(4-Amino-1- piperidinyl)methyl]-N- [3- (phenylmethoxy)phenyl]pyrrolo[2,1- f] [1,2,4]triazin-4-amine | 429.26 | 2.183 (a) |
| 12 | 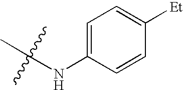 | 5-[(4-Amino-1- piperidinyl)methyl]-N- (4- ethylphenyl)pyrrolo[2,1- f] [1,2,4]triazin-4-amine | 351.28 | 1.64 (a) |
| 13 | 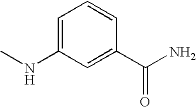 | 3-[[5-[(4-Amino-1- piperidinyl)methyl]pyrrolo [2,1-f] [1,2,4]triazin- 4-yl]amino]benzamide | 366.25 | 0.76 (a) |
| 14 | 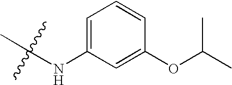 | 5-[(4-Amino-1- piperidinyl)methyl]-N- [3-(1- methylethoxy)phenyl]pyrrolo[2,1- f] [1,2,4]triazin-4-amine | 381.27 | 1.66 (a) |
| 15 | 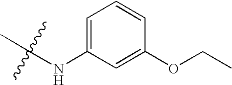 | 5-[(4-Amino-1- piperidinyl)methyl]-N- (3- ethoxyphenyl)pyrrolo[2, 1-f] [1,2,4]triazin-4- amine | 367.26 | 1.44 (a) |
| 16 | 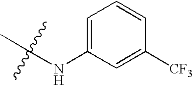 | 5-[(4-Amino-1- piperidmyl)methyl]-N- [3- (trifluoromethyl)phenyl]pyrrolo[2,1- f] [1,2,4]triazin-4-amine | 391.18 | 1.71 (a) |
| 17 | 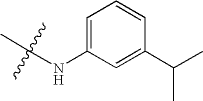 | 5-[(4-Amino-1- piperidinyl)methyl]-N- [3-(1- methylethyl)phenyl]pyrrolo [2,1-f] [1,2,4]triazin- 4-amine | 365.29 | 1.89 (a) |
| 18 | 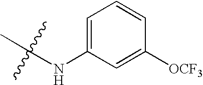 | 5-[(4-Amino-1- piperidinyl)methyl]-N- [3- (trifluoromethoxy)phenyl]pyrrolo[2,1- f] [1,2,4]triazin-4-amine | 407.19 | 1.90 (a) |
| 19 | 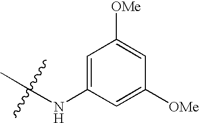 | 5-[(4-Amino-1- piperidinyl)methyl]-N- (3,5- dimethoxyphenyl)pyrrolo [2,1-f] [1,2,4]triazin-4- amine | 383.24 | 1.32 (a) |
| 20 | 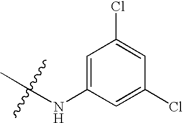 | 5-[(4-Amino-1- piperidinyl)methyl]-N- (3,5- dichlorophenyl)pyrrolo [2,1-f] [1,2,4]triazin-4- amine | 391.13 | 1.85 (a) |
| 21 | 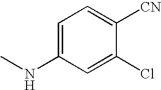 | 4-[[5-[(4-Amino-1- piperidinyl)methyl]pyrrolo [2,1-f] [1,2,4]triazin- 4-yl]amino]-2- chlorobenzonitrile | 382.19 | 1.63 (a) |
| 22 | 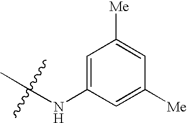 | 5-[(4-Amino-1- piperidinyl)methyl]-N- dimethylphenyl)pyrrolo [2,1-f] [1,2,4]triazin-4- amine | 351.28 | 1.56 (a) |
| 23 | 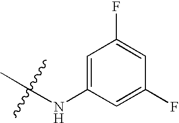 | 5-[(4-Amino-1- piperidinyl)methyl]-N- (3,5- difluorophenyl)pyrrolo [2,1-f] [1,2,4]triazin-4- amine | 359.24 | 1.14 (a) |
| 24 | 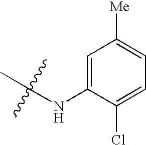 | 5-[(4-Amino-1- piperidinyl)methyl]-N- (2-chloro-5- methylphenyl)pyrrolo[2, 1-f] [1,2,4]triazin-4- amine | 371.21 | 1.38 (a) |
| 25 | 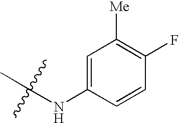 | 5-[(4-Amino-1- piperidinyl)methyl]-N- (4-fluoro-3- methylphenyl)pyrrolo[2, 1-f] [1,2,4]triazin-4- amine | 355.27 | 1.33 (a) |
| 26 | 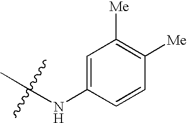 | 5-[(4-Amino-1- piperidinyl)methyl]-N- (3,4- dimethylphenyl)pyrrolo [2,1-f] [1,2,4]triazin-4- amine | 351.28 | 1.58 (a) |
| 27 | 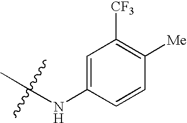 | 5-[(4-Amino-1- piperidinyl)methyl]-N- [4-methyl-3- (trifluoromethyl)phenyl]pyrrolo[2,1- f] [1,2,4]triazin-4-amine | 405.21 | 2.10 (a) |
| 28 | 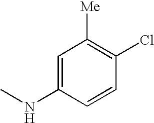 | 5-[(4-Amino-1- piperidinyl)methyl]-N- (4-chloro-3- methylphenyl)pyrrolo[2, 1-f] [1,2,4]triazin-4- amine | 371.21 | 1.74 (a) |
| 29 | 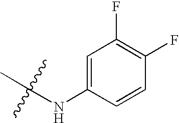 | 5-[(4-Amino-1- piperidinyl)methyl]-N- (3,4- difluorophenyl)pyrrolo [2,1-f] [1,2,4]triazin-4- amine | 359.23 | 1.16 (a) |
| 30 | 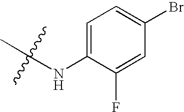 | 5-[(4-Amino-1- piperidinyl)methyl]-N- (4-bromo-2- fluorophenyl)pyrrolo[2, 1-f] [1,2,4]triazin-4- amine | 419.08 | 1.55 (a) |
| 31 | 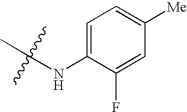 | 5-[(4-Amino-1- piperidinyl)methyl]-N- (2-fluoro-4- methylphenyl)pyrrolo[2, 1-f] [1,2,4]triazin-4- amine | 355.22 | 1.42 (a) |
| 32 | 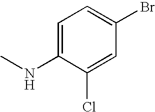 | 5-[(4-Amino-1- piperidinyl)methyl]-N- (4-bromo-2- chlorophenyl)pyrrolo[2, 1-f] [1,2,4]triazin-4- amine | 435.08 | 1.68 (a) |
| 33 | 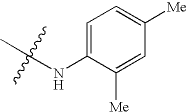 | 5-[(4-Amino-1- piperidinyl)methyl]-N- (2,4- dimethylphenyl)pyrrolo [2,1-f] [1,2,4]triazin-4- amine | 351.24 | 1.36 (a) |
| 34 | 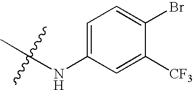 | 5-[(4-Amino-1- piperidinyl)methyl]-N- [4-bromo-3- (trifluoromethyl)phenyl]pyrrolo[2,1- f] [1,2,4]triazin-4-amine | 469.12 | 2.35 (a) |
| 35 | 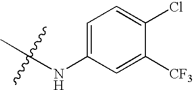 | 5-[(4-Amino-1- piperidinyl)methyl]-N- [4-chloro-3- (trifluoromethyl)phenyl]pyrrolo[2,1- f] [1,2,4]triazin-4-amine | 425.13 | 2.26 (a) |
| 36 | 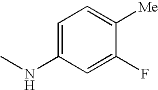 | 5-[(4-Amino-1- piperidinyl)methyl]-N- (3-fluoro-4- methylphenyl)pyrrolo[2, 1-f] ]1,2,4]triazin-4- amine | 355.22 | 1.51 (a) |
| 37 | 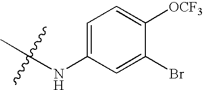 | 5-[(4-Amino-1- piperidinyl)methyl]-N- [3-bromo-4- (trifluoromethoxy)phenyl]pyrrolo[2,1- f] [1,2,4]triazin-4-amine | 485.11 | 2.43 (a) |
| 38 | 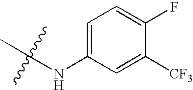 | 5-[(4-Amino-1- piperidinyl)methyl]-N- [4-fluoro-3- (trifluoromethyl)phenyl]pyrrolo[2,1- f] [1,2,4]triazin-4-amine | 409.17 | 1.88 (a) |
| 39 | 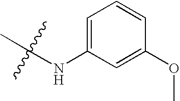 | 5-[(4-Amino-1- piperidinyl)methyl]-N- (3- methoxyphenyl)pyrrolo [2,1-f] [1,2,4]triazin-4- amine, trifluoroacetic acid salt (1:1). | 353 | 1.17 (a) |
| 40 | 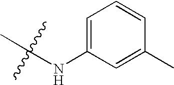 | 5-[(4-Amino-1- piperidinyl)methyl]-N- (3- methylphenyl)pyrrolo[2, 1-f] [1,2,4]triazin-4- amine, trifluoroacetic acid salt (1:1). | 337 | 1.25 (a) |
| 41 | 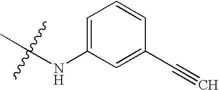 | 5-[(4-Amino-1- piperidinyl)methyl]-N- (3- ethynylphenyl)pyrrolo[2, 1-f] [1,2,4]triazin-4- amine. | 347 | 1.35 (a) |
| 42 | 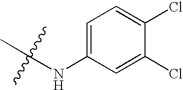 | 5-[(4-Amino-1- piperidinyl)methyl]-N- (3,4- dichlorophenyl)pyrrolo [2,1-f] [1,2,4]triazin-4- amine. | 391 | 1.91 (a) |
| 43 | 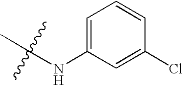 | 5-[(4-Amino-1- piperidinyl)methyl]-N- (3- chlorophenyl)pyrrolo[2, 1-f] [1,2,4]triazin-4- amine. | 357 | 0.75 (a) |

HPLC conditions:

(a): (YMC S5 ODS column 4.6×50 mm, 10-90% aqueous methanol over 4 minutes containing 0.2% H₃PO₄, 3 ml/min, monitoring at 220 nm)

(b): (Chromolith SpeedROD 4.6×50 mm, 10-90% aqueous methanol over 4 minutes containing 0.1% TFA, 4 ml/min, monitoring at 220 nm)

### EXAMPLE 75

### Preparation of Compound 75A (C₆-methyl)

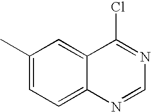

To a mixture of 6-methylquinazolin-4(3H)-one (395 mg, 2.47 mmol), prepared according to literature,¹ in 5 mL of dry toluene, was added i-PrEt₂N (0.8 mL), followed by POCl₃ (3.4 mL). The mixture was heated to 120° C. for 2 hr. After cooling to room temperature, the volatiles were removed under reduced pressure and the residue was dissolved in CH₂Cl₂, washed with cold, half-saturated NaHCO₃ aq. solution and 5% citric acid and dried over anhydrous MgSO₄. Concentration in vacuo gave 415 mg of Compound 75A as a solid. ¹H-NMR (400 MHz, CDCl₃): 9.00 (s, 1H), 8.04 (d, J=1.86 Hz, 1H), 7.98 (d, J=8.61 Hz, 1H), 7.80 (dd, J=1.86 Hz, J=8.61 Hz, 1H), 2.62 (s, 3H).

### Preparation of Compound 75B (C₅-methyl)

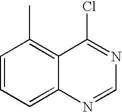

Compound 75B was prepared in an analogous method from 5-methylquinazolin-4(3H)-one (prepared according to literature¹) as a solid. ¹H-NMR (400 MHz, CDCl₃): 8.96 (s, 1H), 7.94 (d, J=8.52 Hz, 1H), 7.80 (dd, J=7.17 Hz, J=8.52 Hz, 1H), 7.51 (d, J=7.17 Hz, 1H), 3.05 (s, 3H).

### Preparation of Compound 75C (C₇-methyl)

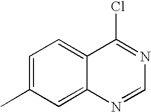

Compound 75C was prepared in an analogous method from 7-methylquinazolin-4(3H)-one (prepared according to literature¹) as a solid. ¹H-NMR (400 MHz, CDCl₃): 8.93 (s, 1H), 8.09 (d, J=8.50 Hz, 1H), 7.78 (s, 1H), 7.50 (d, J=8.50 Hz, 1H), 2.55 (s, 3H).

### EXAMPLE 76

### Preparation of Compound 76A (C₆-methyl)

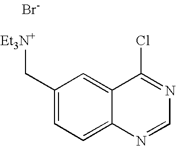

Compound 76A (450 mg) was prepared from 75A (397 mg) as a solid using an analogous method for the preparation of Compound 9. ¹H-NMR (400 MHz, DMSO-d₆): 9.23 (s, 1H), 8.51 (d, J=1.39 Hz, 1H), 8.24 (d, J=8.66 Hz, 1H), 8.19 (dd, J=1.39 Hz, J=8.66 Hz, 1H), 4.84 (s, 2H), 3.26 (q, J=7.16 Hz, 6H), 1.36 (t, J=7.16 Hz, 9H).

### Preparation of Compound 76B (C₅-methyl):

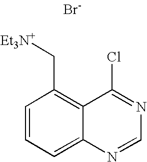

Compound 76B (323 mg) was prepared from 75B (373 mg, yield 43%) as a solid using an analogous method for the preparation of Compound 9. ¹H-NMR (400 MHz, DMSO-d₆): 8.47 (s, 1H), 7.94 (m, 2H), 7.64 (dd, J₁=6.8 Hz, J₂=0.8 Hz, 1H), 5.38 (s, 2H), 3.07 (q, J=7.2 Hz, 6H), 1.27 (t, J=7.2 Hz, 9H).

### Preparation of Compound 76C (C₇-methyl)

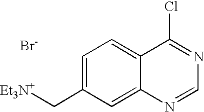

Compound 76C (450 mg) was prepared from 75C (373 mg, yield 60%) as a solid using an analogous method for the preparation of Compound 9. ¹H-NMR (400 MHz, DMSO-d₆): 8.41 (s, 1H), 8.22 (d, J=8.0 Hz, 1H) 7.92 (s, 1H), 7.68 (d, J=8.0 Hz, 1H), 4.71 (s, 2H), 3.24 (q, J=7.1 Hz, 6H), 1.33 (t, J=7.1 Hz, 9H).

### EXAMPLE 77

### Preparation of Compound 77A (C₆-methyl)

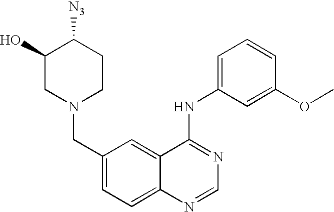

A mixture of Compound 76A (80 mg, 0.15 mmol) and m-methoxyaniline (18 mg, 0.15 mmol) in 1.0 mL of dry CH₃CN was heated to 75° C. for 4.0 hr. After cooling to room temperature, a mixture of (3R,4R)-4-azidopiperidin-3-ol, 47C (23.3 mg, 0.164 mmol) and triethylamine (0.042 mL, 0.298 mmol) in 2 mL of dry DMF was added and the reaction mixture was heated in a microwave oven (60W power setting) at 150° C. for 1.0 hr. After cooling to room temperature, the reaction was diluted with water (10 mL) and extracted with EtOAc (3×20 mL). The combined organic layers were washed with 10% LiCl aq. solution and brine and dried over anhydrous NaSO4. Concentration in vacuo afforded Compound 77A as an oil, which was used in the next reaction step.

### Preparation of Compound 77B (C₅-methyl)

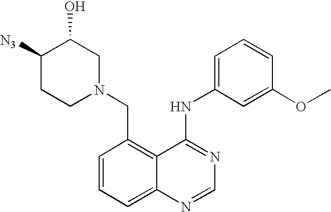

Compound 77B (31 mg) was prepared from 76B (36 mg) as a solid (94% yield) using an analogous method for the preparation of Compound 77A.

### Preparation of Compound 77C (C₇-methyl)

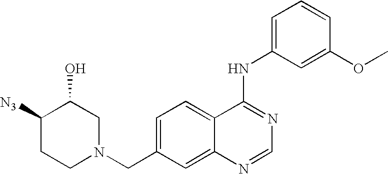

Compound 77C was prepared from 76C as a solid using an analogous method for the preparation of Compound 77A. Compound 77C was a crude product which was used in the next step without further purification. HPLC retention time=1.325 min., M⁺+H=406.

### EXAMPLE 78

### Preparation of Compound 78A (C₆-methyl)

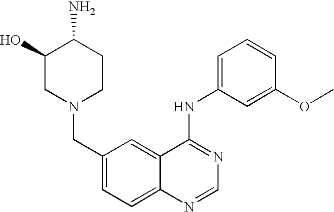

Compound 78A was prepared from Compound 77A in a similar way as Compound 9. Compound 78A is a solid, with an analytical HPLC retention time=1.068 min (Chromolith SpeedROD column 4.6×50 mm, 10-90% aqueous methanol containing 0.1% TFA over 4 minutes, 4 mL/min, monitoring at 254 nm) and a LC/MS M⁺+H=380⁺.

### Preparation of Compound 78B (C₅-methyl)

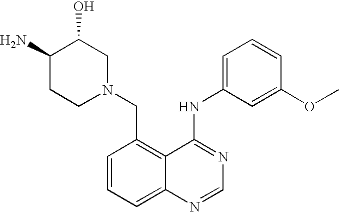

Compound 78B was prepared from Compound 77B in a similar way as Compound 9. Compound 78B is a solid, with an analytical HPLC retention time=1.202 min (Chromolith SpeedROD column 4.6×50 mm, 10-90% aqueous methanol containing 0.1% TFA over 4 minutes, 4 mL/min, monitoring at 254 nm) and a LC/MS M⁺+H =380⁺.

### Preparation of Compound 78C (C₇-methyl)

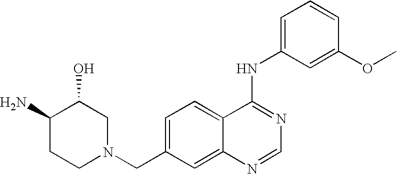

Compound 78C was prepared from Compound 77C in a similar way as Compound 9. Compound 78C is a solid, with an analytical HPLC retention time=1.108 min (Chromolith SpeedROD column 4.6×50 mm, 10-90% aqueous methanol containing 0.1% TFA over 4 minutes, 4 mL/min, monitoring at 254 nm) and a LC/MS M⁺+H=380⁺.

What is claimed is:

1. A process for preparing compound (IV) of the formula 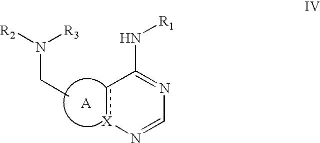
wherein
the A ring is a 5 or 6-membered carbocyclic ring optionally containing one or more heteroatoms in the ring selected from —N—, —O— and —S—, with the proviso that when X is 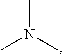
the A ring is a 5-membered aromatic, heterocyclic ring; provided that the compound formed is chemically stable;
R¹ is alkyl or substituted alkyl, aryl or substituted aryl, heteroaryl or substituted heteroaryl or heterocyclyl or substituted heterocyclyl;
R² and R³ are independently hydrogen, alkyl or substituted alkyl, alkoxy or substituted alkoxy, aryl or substituted aryl, heteroaryl or substituted heteroaryl or heterocyclyl or substituted heterocyclyl; or
R² and R³ are taken together with the nitrogen atom to form an optionally substituted 5-7 membered heterocyclic ring optionally containing one or more additional heteroatoms in the ring selected from —N—, —O— and —S—, provided that the compound formed is chemically stable;
X is 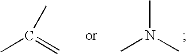
or a pharmaceutically acceptable salt or stereoisomer thereof,
which comprises the steps of
reacting Compound I of the formula 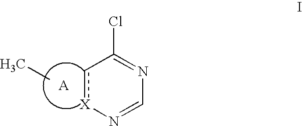
with N-bromosuccinimide in the presence of an activating agent, followed by treatment with a trialkylamine of the formula (R)₃N,
to afford Compound II of the formula 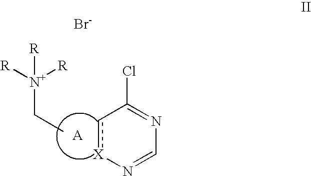
where R is C₁-C₄ alkyl,
which is subsequently reacted with a primary amine of the formula —R¹NH₂,
where R¹ is as defined above,
to afford Compound III of the formula 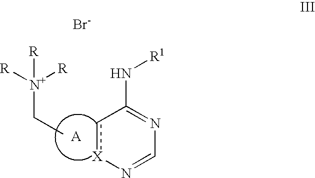
or a salt thereof,
where R¹ is as defined above,
which is reacted with a nucleophile of the formula 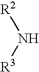
where R² and R³ are as previously defined,
in the presence of a base to afford Compound IV.

2. The process as defined in claim 1 wherein the activating agent is 2′,2′-azobisisobutyronitrile (AIBN) or benzoyl peroxide.

3. The process as defined in claim 1 wherein the trialkylamine is triethylamine.

4. The process as defined in claim 1 wherein the reaction take place in the presence of a solvent selected from the group consisting of acetonitrile, carbon tetrachloride, tetrahydrofuran, chloroform, mixtures of cyclohexanes, chlorobenzenes, dimethylformamide and dimethylacetamide.

5. The process as defined in claim 1 wherein the base used in the final step is triethylamine or diisopropyl ethylamine.

6. A process for preparing Compound 4 of the formula 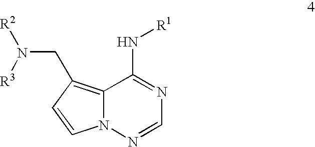
or a pharmaceutically acceptable salt thereof,
where R¹, R² and R³ are as previously defined,
which comprises reacting Compound 1 of the formula 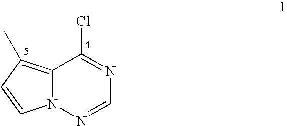
with N-bromosuccinimide in the presence of an activating agent, followed by treatment with a trialkylamine of the formula (R)₃N,
to afford Compound 2 of the formula 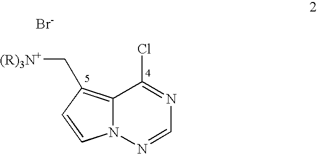
where R is C₁-C₄ alkyl,
which is subsequently reacted with a primary amine of the formula —R¹NH₂,
where R¹ is as defined above,
to afford Compound 3 of the formula 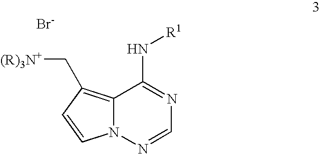
or a HCl salt thereof,
where R¹ is as defined above,
which is reacted with a nucleophile of the formula 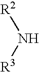
where R² and R³ are as previously defined,
in the presence of a base to afford Compound 4.

7. The process as defined in claim 6 wherein the activating agent is 2′,2′-azobisisobutyronitrile (AIBN) or benzoyl peroxide.

8. The process as defined in claim 6 wherein the trialkylamine is triethylamine.

9. The process as defined in claim 6 wherein the reaction take place in the presence of a solvent selected from the group consisting of acetonitrile, carbon tetrachloride, tetrahydrofuran, chloroform, mixtures of cyclohexanes, chlorobenzenes, dimethylformamide and dimethylacetamide.

10. The process as defined in claim 6 wherein the base used in the final step is triethylamine or diisopropyl ethylamine.

11. A process for preparing Compound 8 of the formula 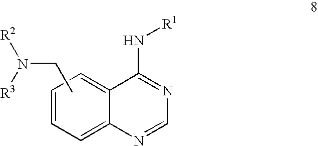
or a pharmaceutically acceptable salt thereof,
where R¹, R² and R³ are as previously defined,
which comprises reacting Compound 5 of the formula 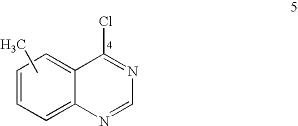
with N-bromosuccinimide in the presence of an activating agent, followed by treatment with a trialkylamine of the formula (R)₃N,
to afford Compound 6 of the formula 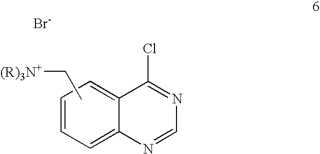
where R is C₁-C₄ alkyl,
which is subsequently reacted with a primary amine of the formula —R¹NH₂,
where R¹ is as defined above, to afford Compound 7 of the formula 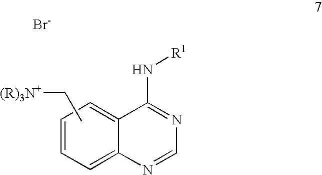
or a HCl salt thereof,
where R¹ is as defined above,
which is reacted with a nucleophile of the formula 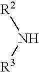
where R² and R³ are as previously defined,
in the presence of a base to afford Compound 8.

12. The process as defined in claim 11 wherein the activating agent is 2′,2′-azobisisobutyronitrile (AIBN) or benzoyl peroxide.

13. The process as defined in claim 11 wherein the trialkylamine is triethylamine.

14. The process as defined in claim 11 wherein the reaction take place in the presence of a solvent selected from the group consisting of acetonitrile, carbon tetrachloride, tetrahydrofuran, chloroform, mixtures of cyclohexanes, chlorobenzenes, dimethylformamide and dimethylacetamide.

15. The process as defined in claim 11 wherein the base used in the final step is triethylamine or diisopropyl ethylamine.

16. Compound 9 of the formula 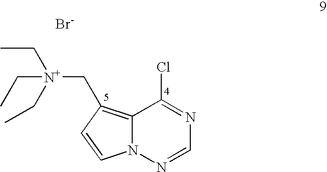
or a salt thereof.

17. A pharmaceutical composition comprising one or more compound prepared by the process of claim 1 and a pharmaceutically acceptable carrier therefor.

18. A pharmaceutical composition comprising one or more compound prepared by the process of claim 1 in combination with a pharmaceutically acceptable carrier and one or more other anti-cancer or cytotoxic agent.
